(19) United States
(12) Patent Application Publication
(10) Pub. No.: US 2015/0119845 A1
(43) Pub. Date: Apr. 30, 2015

(54) SECURING DEVICE FOR MEDICAL LINES

(71) Applicants: Steve A. Collins, Blue Ridge, GA (US); Cynthia G. Collins, Blue Ridge, GA (US)

(72) Inventors: Steve A. Collins, Blue Ridge, GA (US); Cynthia G. Collins, Blue Ridge, GA (US)

(21) Appl. No.: 14/061,834

(22) Filed: Oct. 24, 2013

(51) Int. Cl. A61M 25/02

(52) U.S. Cl. CPC A61M 25/02; A61M 2025/0253; A61M 2025/024
USPC 604/500; 604/180

(57) ABSTRACT

Disclosed are various embodiments of medical line securing devices and methods of using the medical line securing device. According to various embodiments, the medical line securing device comprises a bandage that comprises at least one releasable locking device configured to detachably attach to at least one intravenous extension tube. In one embodiment, the intravenous extension tube may be coupled to a source intravenous line on one end and an access device on the other end. The releasable locking device may be configured to detach the intravenous extension tube when a pre-defined force is exerted on the intravenous extension tube.

### BACKGROUND

Intravenous line and catheter complications may occur if an intravenous line is positioned incorrectly or becomes dislodged from a vein. For instance, if pressure is not applied firmly when an intravenous line is removed, blood may leak out of the vein and build up in the tissues resulting in pain, swelling, infection, and blood clots. The complications associated with intravenous therapy may result in not only a rise in healthcare costs from prolonged hospitalization and an extended use of antibiotic therapy, but also surgical intervention and possibly even death. Intravenous therapy complications happen regularly as a result of patients accidentally dislodging an intravenous line or catheter from the vein. Thus, these lines and catheters need to be secured to the patient to avoid such accidental removal from the vein.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout several views.

FIG. 1 is a drawing of a substance delivery system according to various embodiments of the present disclosure.

FIG. 2A is a drawing of one embodiment of an access device.

FIG. 2B is a drawing of another embodiment of an access device.

FIG. 3 is a drawing of an embodiment of an intravenous extension tube.

FIGS. 4A-4B are drawings of adhesive backings for the access device of FIG. 2A comprising releasable locking devices.

FIGS. 5A-5B are drawings of adhesive backings for the access device of FIG. 2B comprising releasable locking devices.

FIGS. 6A-6B are drawings illustrating an embodiment of an intravenous line securing system.

FIGS. 7A-7B are drawings illustrating an embodiment of a multi-port line securing system.

FIGS. 8A-8C are drawings showing perspective views of one embodiment of the releasable locking device.

FIGS. 9A-9H are drawings showing alternative embodiments of the releasable locking device.

FIGS. 10A-B are drawings showing alternative embodiments of the intravenous line securing system depicted in FIGS. 6A-6B.

FIG. 11 is a flowchart illustrating one example of functionality implemented by a user of a substance delivery system comprising the line securing system according to various embodiments of the present disclosure.

### DETAILED DESCRIPTION

The present disclosure relates to securing intravenous lines and central venous lines. In some embodiments, the systems include an intravenous extension tube with a specially designed attachable portion. One end of the intravenous extension tube is affixed to a source intravenous tubing that receives a substance from a solution delivery bag. The other end of the intravenous extension tube is affixed to a target intravenous tubing. The intravenous extension tube may be configured to attach to a releasable locking device of an adhesive backing attachable to a patient. A solution, or other content, may travel from the solution delivery bag, through the source intravenous tubing, through the intravenous extension tube, and through the target intravenous tubing into an access device that injects the solution into the patient.

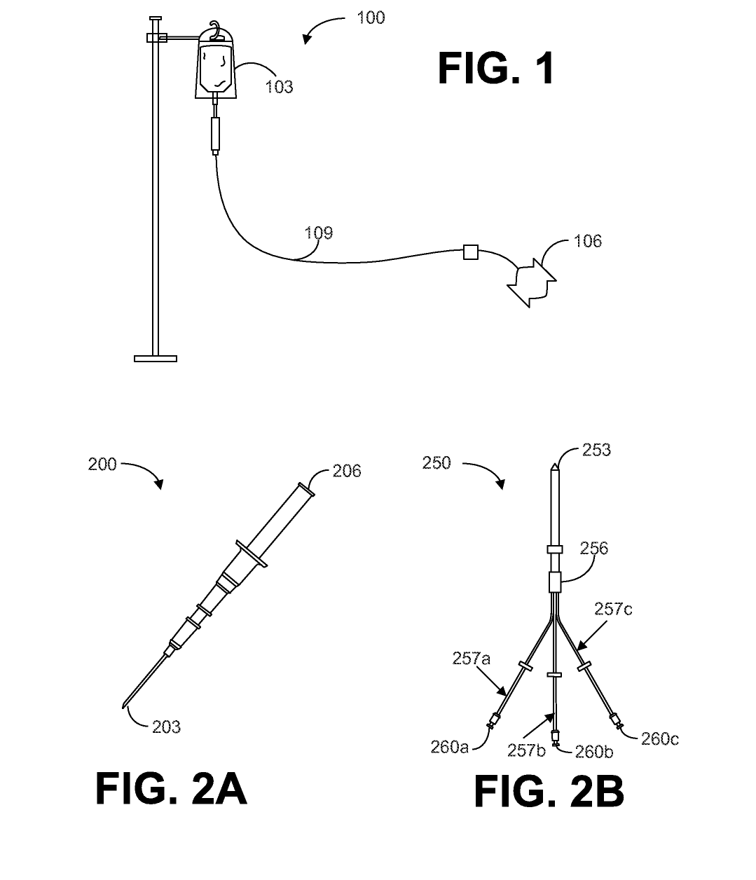

FIG. 1 is a drawing of a substance delivery system according to various embodiments of the present disclosure. The substance delivery system 100 comprises at least one substance delivery bag 103 connected to an adhesive backing 106 by means of one or more lines 109. The lines 109 may be intravenous tubing, electrical lines, drainage tubes, or lumen tubing. The substance delivery bag 103 may comprise one or more pre-formed concentrated solutions that may be diluted for intravenous injection. For example, the substance delivery bag 103 may contain fluids containing electrolytes to be delivered through the line 109 to a dehydrated patient. The solutions from the substance delivery bag 103 is to be injected into the vein of a patient at an injection site. Traditionally, the line 109 may be stabilized at the injection site by using sutures, tunnelers, wings, anchor pads, clamps, injection caps, and/or any Statlock® stabilization device. The line 109 may combine solutions from the substance delivery bag 103. At least a portion of the line 109 may be coupled to the adhesive backing 106. The adhesive backing 106 may also be partially attached to an infusion member configured to inject the solutions into the patient at the injection site. As may be appreciated, the injection sites include but are not limited to, the hand, arm, neck or leg of a patient. A patient may be a human, an animal, any living being, or an electrical device, including but not limited to, a robot, vehicle, machine, or computer. The line 109 may be an electrical cable or wire, and the injection site may be a location of the electrical device, including, but not limited to, a robot, vehicle, machine, or computer. As may be appreciated, the solution delivery bag mentioned throughout this disclosure may also represent a central line hub.

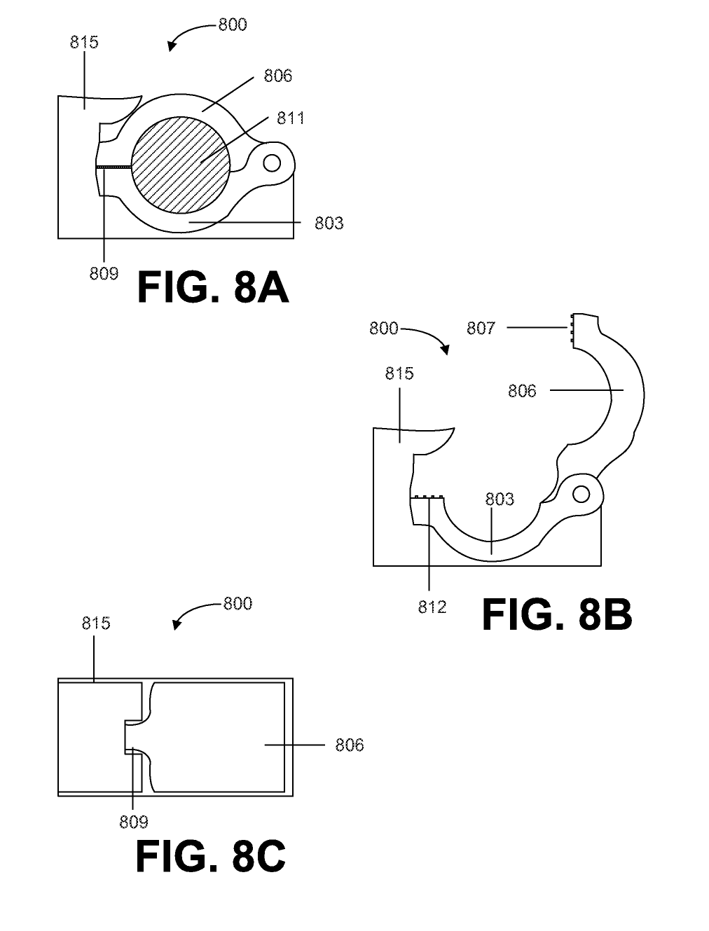

In one embodiment of the present disclosure, an intravenous extension tube may be disposed on an end of the line 109 and the intravenous extension tube may be attached to a releasable locking device on the adhesive backing 106. Examples of such a releasable locking device are further described below in relation to FIGS. 8A-C and 9A-F. In such an embodiment, the solutions may travel from the substance delivery bag 103, through the line 109, through the intravenous extension tube, through the access device, and into the patient.

In another embodiment, the intravenous extension tube may be displaced in between a first portion of the line 109 and a second portion of the line 109. The intravenous extension tube may be coupled to a releasable locking device of the adhesive backing 106. The first portion of the line 109 may channel the solution from the substance delivery bag 103. The second portion of the line 109 may be coupled to an access device or an infusion member configured to inject the solutions into the patient at the injection site.

FIG. 2A is a drawing of one embodiment of an access device 200. As may be appreciated, an access device 200 may be, but is not limited to, a drainage device, an imaging device, an epidural line, a dialysis catheter, a neonatal line, a pediatric line, an intravenous line, or any line or device that may be inserted directly into a patient's body. A drainage device may be a surgical drain tube used to remove pus, blood, or other fluids from a wound. As such, a drainage device may be inserted anywhere into a patient's body where there is a need for such drainage. Examples of drain tubes include, but are not limited to, a nephrostomy tube, a thoracic tube, a Jackson-Pratt® drain, a Penrose drain, a negative pressure wound therapy drain, a redivac drain, a pigtail drain, a davol, a wound managing drain, or any drainage tube that may be inserted into the patient's body.

For example, a patient suffering from internal hydrocephalus, also known as water on the brain, has an accumulation of fluids in the ventricles of the brain. The internal hydrocephalus may be successfully treated by placing a drainage tube between the brain ventricles and abdominal cavity to eliminate the high intracranial pressure. In such a case, the patient may suffer from fatal injury if the drainage tube is displaced at any time during the drainage. As such, the drainage tube may be secured by using the securing device for medical lines disclosed herein.

As another example, a patient with kidney failure may be treated using a nephrostomy tube that allows for the urinary diversion directly from the upper part of the urinary system. The nephrostomy tube may be inserted into the patient at a nephrostomy insertion site created between the kidney and the skin. Similar to the example above, the patient may suffer from infection or other serious harm if the nephrostomy tube is dislodged from the nephrostomy insertion site. The nephrostomy tube may be secured using the securing device for medical lines disclosed herein. Similarly, any patient receiving treatment using a drainage tube may utilize the securing device to secure the drainage tube to the patient at the insertion site to prevent injury from dislodging of the drainage tube.

An intravenous line, as illustrated in FIG. 2A, may be, but is not limited to an arterial, a central, a midline, or a peripheral intravenous line. The intravenous line access device 200 may comprise, for example, a needle 203, a tube inlet 206, and other components. The needle 203 may be configured to inject into a vein of a patient. In one embodiment, the tube inlet 206 is coupled to the intravenous extension tube. In such an embodiment, the tube inlet 206 is configured to receive solutions directly from the intravenous extension tube and then inject the solutions into the patient. In another embodiment, the tube inlet 206 may be coupled to the second portion of a line 109 (FIG. 1). Similar to the embodiment discussed above in relation to FIG. 1, the solutions may travel through the intravenous extension tube through the second portion of the line 109 before traveling through the tube inlet 206 and the needle 203.

FIG. 2B is a drawing of another embodiment of an access device. More specifically, FIG. 2B is a drawing of a multi-port indwelling catheter 250. A multi-port indwelling catheter 250 may be a central venous catheter. Such a central venous cathether may be a catheter placed into a large vein in the neck (internal jugular vein), chest (subclavian vein or axillary vein), or groin (femoral vein). The central venous catheter may be used to administer medication or solutions, obtain blood tests, and directly obtain cardiovascular measures such as, but not limited to, the central venous pressure. One embodiment of a central venous catheter may use a non-tunneled catheter that is fixed in a place at the insertion site, with the catheter and attachments protruding directly. Non-tunneled catheters may include Quinton catheters.

Another embodiment of a central venous catheter may use a tunneled catheter. Tunneled catheters are passed under the skin from the insertion site to a separate exit site, where the catheter and its attachments emerge from underneath the skin. The exit site is typically located in the chest, making the access ports less visible than if they were protruding directly from the neck. Passing the catheter under the skin helps to prevent infection and provides stability. Tunneled catheters may include Hickman catheters and Groshong catheters.

Another embodiment of a central venous catheter may use an implanted port. A port is similar to a tunneled catheter but is left entirely under the skin. Medicines are injected through the skin into the catheter. Some implanted ports contain a small reservoir that can be refilled in the same way. After being filled, the reservoir slowly releases the medicine into the bloodstream. Yet another embodiment of a central venous catheter is a peripherally inserted central catheter line, which is a central venous catheter inserted into a vein in the arm rather than a vein in the neck or chest.

FIG. 2B illustrates a multi-port indwelling catheter 250 that may couple to three lines. The multi-port indwelling catheter 250 may comprise a central needle 253, a connector 256, intermediaries 257a-c, and line ports 260a-c. Each of the intermediaries may represent an intravenous tube, an electrical line, and/or a single or multi lumen tubing that is displaced between an intravenous extension tube and the connector 256. Each of the line ports may represent, for example, a proximal port, a medial port, or a distal port. Each of the line ports may be coupled to a separate intravenous tubing or electric line.

The central needle 253 may be inserted into the patient at an injection site. The line ports 260a-c may attach to a respective intravenous extension tube. Each intravenous extension tube may comprise an attachable portion configured to attach to a releasable locking device of an adhesive backing secured to a patient. In one embodiment, each intravenous extension tube may be connected to the respective line port 260a-c at one end and the connector 256 at another end of the intravenous extension tube. In an alternative embodiment, one intravenous extension tube may be disposed at the connector 256. In yet another embodiment, one end of the intravenous extension tube may be connect to the respective line port 260a-c and the other end of the intravenous extension tube may be connected to lines connected to or channel fluids from at least a portion of a central hub.

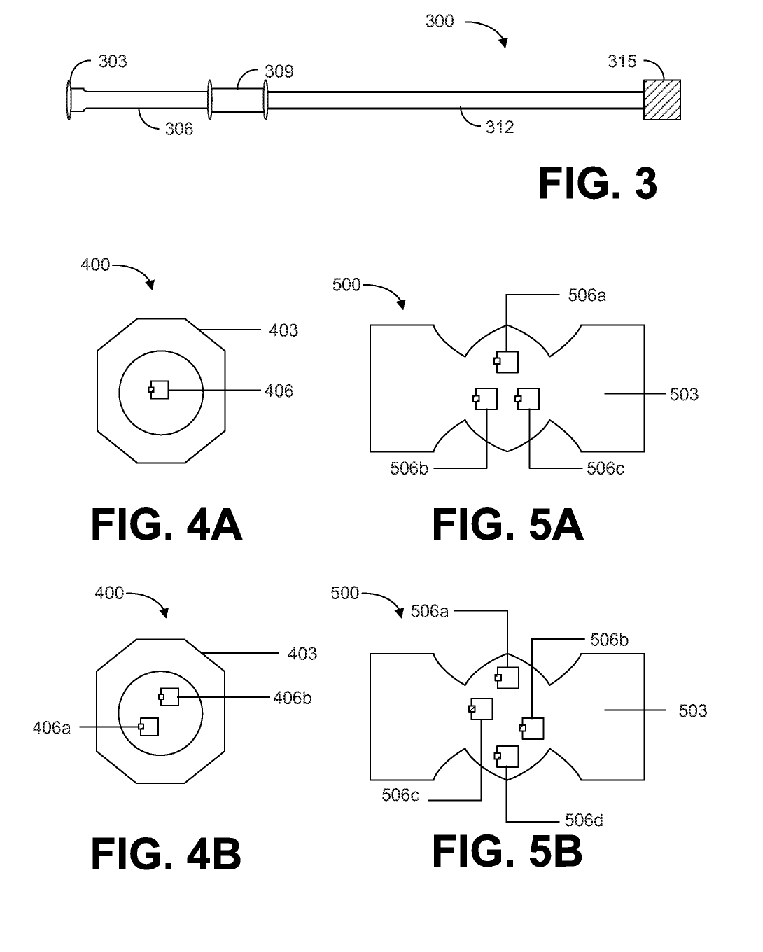

FIG. 3 is a drawing of an embodiment of an intravenous extension tube 300. The intravenous extension tube 300 comprises an inlet connector 303, a first section 306, an attachable portion 309, a second section 312, and an outlet connector 315. The inlet connector 303 may couple to a source intravenous line that channels solutions to be delivered to the patient. Alternatively the inlet connector 303 may couple to an electric cable configured to monitor equipment of the solution delivery system and/or obtain cardiovascular measures of a patient. The inlet connector 303 may couple to a first section 306 of the intravenous extension tube 300. The first section 306 of the intravenous extension tube 300 may couple to the attachable portion 309. The attachable portion 309 may be configured to attach to a releasable locking device of an adhesive backing to be secured to a patient. The attachable portion 309 may be coupled to the second section 312 of the intravenous extension tube 300. In one embodiment, the second section 312 may be made of a more rigid material than the first section 306. In another embodiment, the first section 306 may be made of a more rigid material than the second section 312. In yet another embodiment, the first section 306 may be of the same rigidness of the second section 312. The second section 312 may be coupled to an outlet connector 315. The outlet connector 315 may be configured to couple to a portion of the intravenous tubing or medical line. Alternatively, the outlet connector 315 may couple directly to the access device. According to the various embodiments, the intravenous extension tube 300 may be about eight inches long.

As an illustrative example, the intravenous extension tube 300 may be used in connection with the access device described in FIG. 2A. The inlet connector 303 may connect to a line of a substance delivery system. In one embodiment, the outlet connector 315 may connect to the tube inlet 206 of FIG. 2A. In another embodiment, the outlet connector 315 may connect anywhere on the access device 200 of FIG. 2A. The outlet connector 315 may alternatively connect to sutures, tunnelers, wings, anchor pads, clamps, injection caps, and/or any Statlock® stabilization device used on or in proximity to the insertion site. The intravenous extension tube 300 may serve as an intermediary between the line and the access device 200 represented in FIG. 2A.

As another illustrative example, a plurality of intravenous extension tubes 300 may be used in connection with the multi-port indwelling catheter 250 described in FIG. 2B. Each of the inlet connectors 303 may respectively connect to a line of a substance delivery system. In one embodiment, each of the outlet connectors 315 may be coupled to one of the line ports 260a-c of FIG. 2B. In another embodiment, the outlet connector 315 may be coupled to the connector 256 of FIG. 2B. The intravenous extension tube 300 may serve as an intermediary between the needle 253 (FIG. 2B) and the line 109 (FIG. 1) to be injected into the patient and each of the lines.

As an example of the functionality of the intravenous extension tube 300, a patient receiving fluids from a substance delivery system, as described in FIG. 1, may have the access device 200 or multi-port indwelling catheter 250 from FIGS. 2A or FIG. 2B secured to the patient's body using an adhesive bandage comprising a releasable locking device. The intravenous extension tube 300 may be located in between the access device and the line 109 from FIG. 1. Suppose a patient accidentally knocks down the line 109 from FIG. 1, then the releasable locking device will detach from the intravenous extension tube 300, preventing the needle 253 (FIG. 2B) of the multi-port indwelling catheter 250 (FIG. 2B) from dislodging inside the patient.

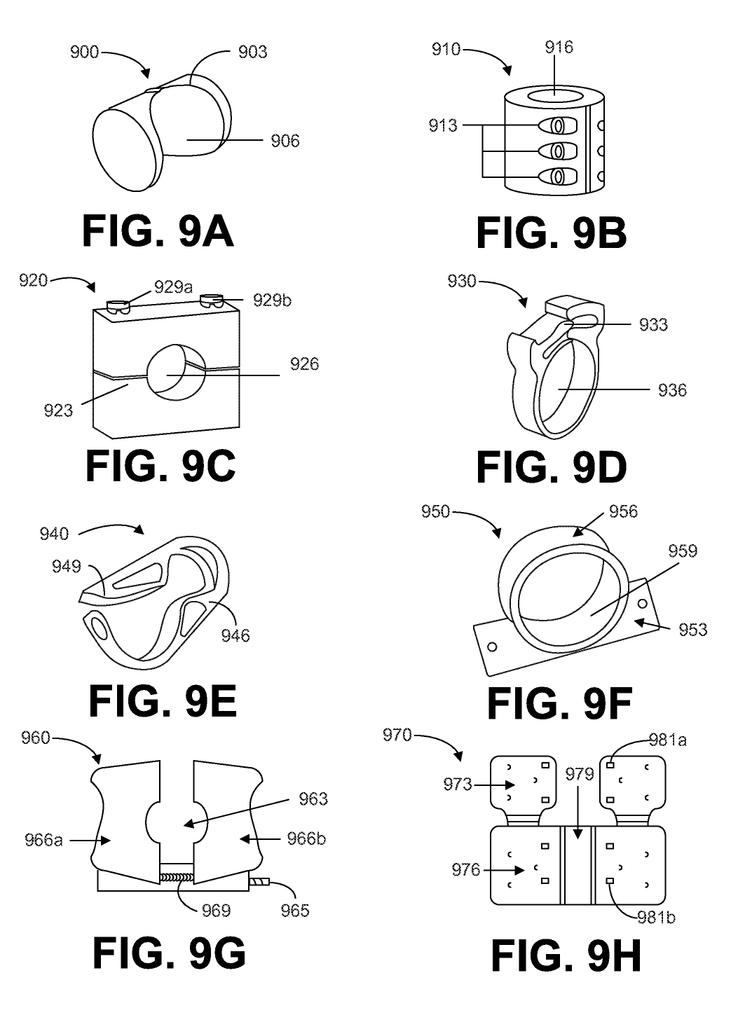

FIGS. 4A-4B are drawings of adhesive backings for intravenous lines that comprise releasable locking devices. FIG. 4A illustrates an adhesive backing 400 comprising an adhesive portion 403 and one releasable locking device 406. In one embodiment, the adhesive backing 400 may be configured to be secured to the patient in proximity to an injection site using the adhesive portion 403. As may be appreciated, the adhesive backing may sterile or non-sterile. The adhesive backing may also be transparent and further comprise a gauze pad. The releasable locking device 406 may be configured to detachably attach to the attachable portion of an intravenous extension tube, as described above in FIG. 3 in relation to the attachable portion 309. Examples of structures of the releasable locking device 406 that may be employed in the non-limiting example of FIG. 4A are described further in FIGS. 8 and 9. As may be appreciated, the adhesive backing 400 may be made of any material that may be attached together tightly around patient at a location of the patient's body.

FIG. 4B illustrates the adhesive backing 400 comprising the adhesive portion 403 and two releasable locking devices 406a-b. Each of the releasable locking devices 406a-b may be configured to attach to an intravenous extension tube of a two-port indwelling catheter line similar to the releasable locking device 406 described in FIG. 4A. As illustrated in FIG. 4B, the releasable locking devices 406a-b may be positioned on the adhesive backing in a staggered manner such that an intravenous extension tube 300 (FIG. 3) may pass through each one of the releasable locking device 406a-b. Alternatively, the releasable locking devices 406a-b may be positioned on the adhesive backing in a matrix form, where each of the releasable locking devices 406a-b are positioned parallel and perpendicular to one another.

FIGS. 5A-5B are drawings of adhesive backings for multi-port indwelling catheters comprising a plurality of releasable locking devices. FIG. 5A illustrates an adhesive backing 500 comprising an adhesive portion 503 and three releasable locking devices 506a-c. The three releasable locking devices 506a-c may be configured to detachably attach to the attachable portions of a trilumen indwelling catheter, similar to the one shown in FIG. 2B. The releasable locking devices 506a-c of the adhesive backing 500 may operate similar to the releasable locking device 406 described above in relation to FIG. 4A.

FIG. 5B illustrates the adhesive backing 500 comprising the adhesive portion 503 and four releasable locking devices 506a-d. Each of the releasable locking devices 506a-d may be configured to attach to an intravenous extension tube of a multi-port indwelling catheter line in a manner similar to the releasable locking device 406 described in FIG. 4A. For example, suppose a patient is receiving intravenous therapy from a substance delivery system using a four-lumen indwelling catheter. In such a case, the patient may receive an injection at the injection site, and four lines may be protruding from the injection. An intravenous extension tube 300 (FIG. 3) may be displaced in between a portion of each of the four lines in proximity to the injection site. Specifically, each of the four releasable locking devices 506a-d of the adhesive backing 500 may attach an attachable portion of each of the intravenous extension tubes connected a corresponding one of the four lines protruding from the patient. The adhesive backing 500 may hold the intravenous extension tubes while being secured to the patient in proximity to the injection site. The line securing system reflected by this example functions to protect the patient from enduring any injury as a result of accidently dislodging any of the lines or tubing that may be connected to the patient.

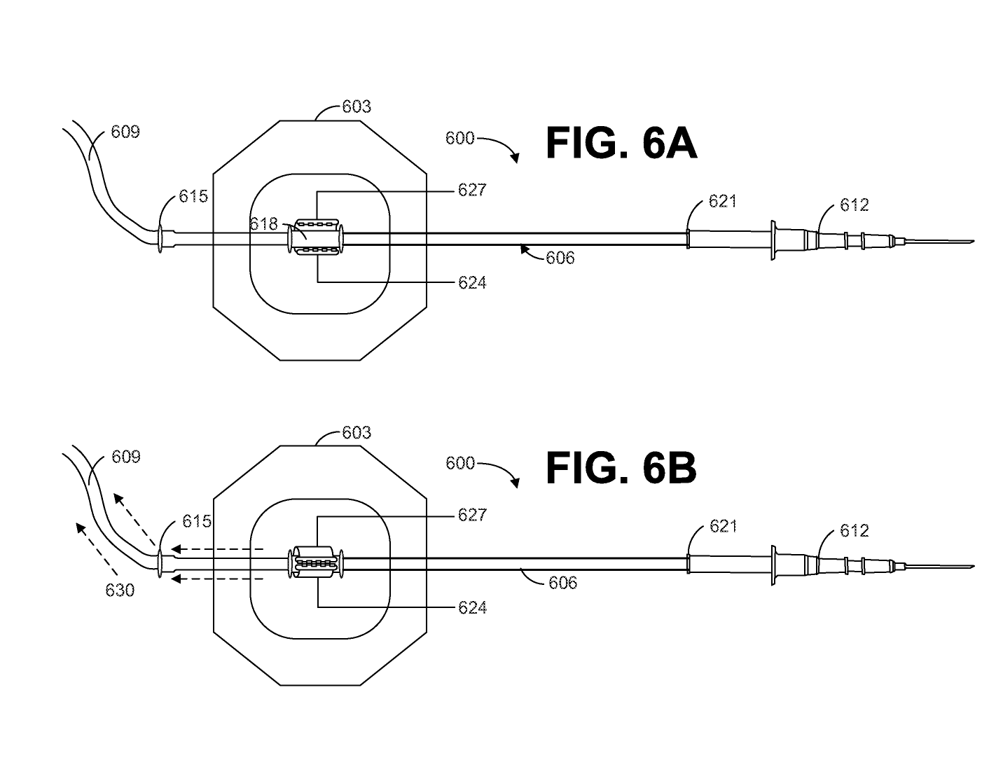

FIG. 6A illustrates an embodiment of an intravenous line securing system 600. The intravenous line securing system 600 comprises an adhesive backing 603, an intravenous extension tube 606, a source intravenous line 609, and an infusion member 612. Similar to the intravenous extension tube 300 described in FIG. 3, the intravenous extension tube 606 may comprise an inlet connector 615, an attachable portion 618, and an outlet connector 621. The source intravenous line 609 may be coupled to the inlet connector 615. The infusion member 612 may be coupled to the outlet connector 621.

The adhesive backing 603 comprises at least one releasable locking device 406 (FIGS. 4A-B) according to various embodiments. The releasable locking device 406 may comprise a lower semicircular arm 624, an upper semicircular arm 627, wherein the upper semicircular arm 627 and the lower semicircular arm 624 are hingedly interconnected at one end and having interlocking portions at the other end. An aperture located in between the lower semicircular arm 624 and the upper semicircular arm 627 may be employed to detachably attach to the attachable portion 618 of the intravenous extension tube 606. The adhesive backing 603 may be configured to secure to a patient in proximity to and/or on the injection site. The source intravenous line 609 may be configured to channel solutions, or other content, to be directed to the patient at the injection site.

FIG. 6B illustrates one example of the functionality of the intravenous line securing system 600. Similar to the embodiment of the system described in FIG. 6A, The intravenous line securing system 600 comprises an adhesive backing 603, an intravenous extension tube 606, a source intravenous line 609, and an infusion member 612. Similar to the intravenous extension tube 300 described in FIG. 3, intravenous extension tube 606 may comprise an inlet connector 615 and an outlet connector 621, wherein the source intravenous line 609 is configured to couple to the inlet connector 615. The infusion member 612 may be coupled to the outlet connector 621.

As above in FIG. 6A, the adhesive backing 603 comprises at least one releasable locking device 406 (FIGS. 4A-B). The releasable locking device 406 may be structured similar to the releasable locking device 406 described in FIG. 6A. As may be appreciated, the releasable locking device 406 may be structured in any way such that the releasable locking device 406 may lock and unlock to the attachable portion of the intravenous extension tube 606. Further descriptions of embodiments of the releasable locking device 406 are described below in relation to FIG. 9.

Next, a description of the operation of various components of the intravenous line securing system 600 is provided. In one embodiment, if a force 630 is exerted upon the intravenous extension tube 606 and/or the source intravenous line 609, then the releasable locking device may be configured to detach from the attachable portion of the intravenous extension tube 606 to prevent the infusion member 612 from dislodging in the patient. The line securing system 600 may increase dwell times and decrease complications in the patient. For example, suppose a patient yawns and accidentally nudges or knocks down the source intravenous line. In this embodiment, the releasable locking device is configured to detach and thereby prevent the needle in the patient from dislodging and injuring the patient.

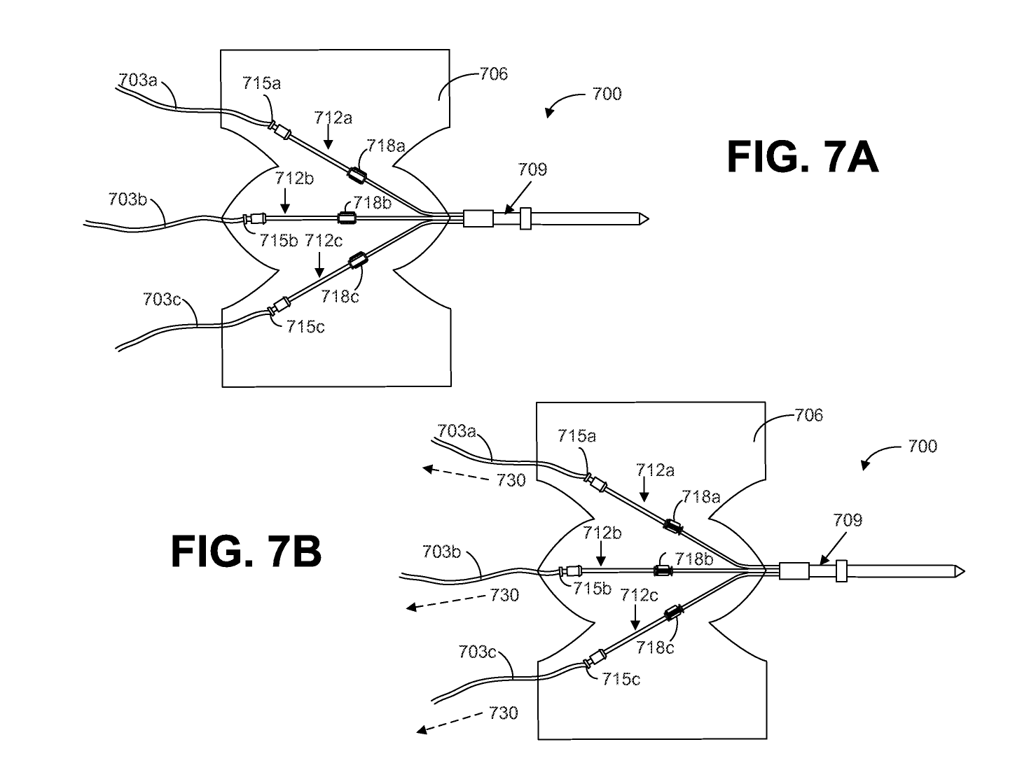

FIG. 7A illustrates an embodiment of a multi-port line securing system 700 comprising three lines 703a-c, an adhesive backing 706, and infusion member 709. The lines 703a-c may be intravenous lines or electric lines configured to monitor equipment or obtain cardiovascular readings, or other readings of the patient. The patient may be a human, an animal, any living being, or an electrical device, including but not limited to, a robot, vehicle, machine, or computer. The adhesive backing 706 is configured to attach to the patient. The infusion member 709 is configured to inject solutions into the patient's veins.

The adhesive backing comprises three releasable locking devices 506 (FIGS. 5A-B), each configured to detachably attach to an attachable portion of an intravenous extension tube 712a-c. Each of the three lines 703a-c respectively couple to three ports 715a-c. Each of the three ports 715a-c may be attached to one end of each intravenous extension tube 712a-c. The other end of the intravenous extension tube 712a-c may be attached to a portion of the infusion member 709. The releasable locking device 718a-c may operate similar to the releasable locking devices described in FIGS. 6A-B.

FIG. 7B illustrates one example of the functionality of the multi-port line securing system 700. Similar to the embodiment of the system described in FIG. 6A, the multi-port line securing system 700 comprises three lines 703a-c, an adhesive backing 706, and infusion member 709. The multi-port line securing system 700 is structured similar to the multi-port line securing system 700 describe in relation to FIG. 7A.

Next, a description of the operation of various components of the multi-port line securing system 700 is provided. In one embodiment, if a force 730 is exerted upon the intravenous extension tube 712a-c and/or the source intravenous line 703a-c, then the releasable locking devices 718a-c may be configured to detach from the attachable portion of the intravenous extension tube 712a-c to prevent the infusion member 709 from dislodging in the patient.

In one embodiment, the multi-port line securing system 700 may be configured such that only the respective releasable locking device 718a-c securing the dislodged source intravenous line 703a-c may be released. Alternatively, the multi-port line securing system 700 may be configured such that all the releasable locking devices 718a-c on the adhesive back 706 may be detached if one or more of the lines 703a-cand/or one or more of the intravenous extension tubes 712a-c have been dislodged from its original position.

According to some embodiments, the line securing system 600 (FIGS. 6A-6B), and the multi-port line securing system 700 may be pre-configured to respond to a specific pre-determined force. For example, suppose the line securing system 600 is built to secure an arterial line. In such a case, the line securing system 600 may be pre-configured to automatically detach at a specific amount of force that the line securing system 600 recognizes as creating a risk of dislodging the arterial line from the patient. As another illustrative example, suppose the multi-port line securing system 700 is built to secure a triple port central venous catheter. In such a case, a small amount of force may be enough to cause a risk of dislodgment or infection in the patient because the catheter may be inserted at sensitive points at the chest or neck. The multi-port line securing system 700 may be pre-configured to automatically detach at a minute amount of force that the multi-port line securing system 700 recognizes as creating a risk to the patient. Depending on the type of access device 315 (FIG. 3) inserted into the patient, the line securing system 600 or the multi-port line securing system 700 may require different amounts of force the detach the releasable locking device from the attachable portion of the intravenous extension tube.

A pre-defined force may be determined for each releasable locking device of the line securing system 600 or the multi-port line securing system 700. The pre-defined force measures the amount of force required to detach the releasable locking device from the intravenous extension tube. The pre-defined force may be measured by conducting experiments using intravenous therapy devices or drainage devices. The experiments may determine how much force is necessary to dislodge the device from the patient and/or how much force is necessary to injure the patient. The pre-defined force may be any amount of force that signals the releasable locking device to detach from the intravenous extension tube to prevent harm to the patient. As described above, different intravenous or drainage treatments require different amounts of force to trigger the detaching of the releasable locking device. As such, each releasable locking device may be manufactured based on the pre-defined force for a specific type of treatment.

According to various embodiments, the line securing system 600 and the multi-port line securing system 700 may be configured to have an adjustable amount of pre-defined force configurable, for example, by a nurse or other medical practitioner. For example, the medical practitioner may adjust the strength of the releasable locking device depending on the insertion site of the patient. For example, suppose the medical practitioner is using the line securing system 600 for an arterial line therapy by inserting a needle into an artery in the chest of the patient. The medical practitioner may recognize the fatal danger associated with any slight dislodgment of the arterial needle in the patient's chest. Then the medical practitioner may adjust the strength of the releasable locking device down such that the releasable locking device detaches upon the exertion of a small amount of force anywhere on the line securing system 600.

FIGS. 8A-8C shows perspective views of various embodiment of the releasable locking device 800. More specifically, FIG. 8A shows an embodiment of a locked releasable locking device 800. The releasable locking device 800 may comprise a lower semicircular arm 803, an upper semicircular arm 806, wherein the upper semicircular arm 806 and the lower semicircular arm 803 are hingedly interconnected at one end and having interlocking portions 809 at the other end. An aperture 811 in between the lower semicircular arm 803 and the upper semicircular arm 806 may be employed to detachably attach to the attachable portion of the intravenous extension tube upon exertion of a pre-defined configurable force on the line, and a latch 815 to further secure the interlocking portions 809.

FIG. 8B shows an embodiment of an unlocked releasable locking device 800. In this embodiment, the releasable locking device 800 comprises the same upper semicircular arm 806, lower semicircular arm 803, first interlocking portion 807, second interlocking portion 812, and latch 815. This releasable locking device 800 is structured similar to the one described in FIG. 8A. In one embodiment, an intravenous extension tube may rest upon the lower semicircular arm 803 and be attached to the releasable locking device 800 by lowering the upper semicircular arm 806 and interlocking the first interlocking portion 807 to the second interlocking portion 812. Finally, the intravenous extension tube may be further locked into the releasable locking device 800 by securing the latch 815.

FIG. 8C shows a top view of the releasable locking device shown in FIGS. 8A and 8B. As illustrated in FIG. 8C, the releasable locking device 800 top view comprises the latch 815 to the left, the interlocking portion 809 in the middle, and the upper semicircular arm 806 to the right.

FIGS. 9A-9F show alternative embodiments of the releasable locking device. In one embodiment, the releasable locking device 900 of FIG. 9A may comprise a base 903 configured to attach to an adhesive backing. The base 903 may comprise a holder 906 configured to receive and secure the attachable portion of the intravenous extension tube. The holder 906 may also be configured to detach the intravenous extension tube when a force is applied to the intravenous extension tube and/or a source intravenous line connected to the intravenous extension tube.

The releasable locking device 910 of FIG. 9B may comprise at least one lock 913 and an aperture 916. The releasable locking device 910 may be coupled to an adhesive backing. The aperture 916 may receive an intravenous extension tube. The lock 913 may further secure the intravenous extension tube to the releasable locking device 910. The releasable locking device 910 may be configured to release the intravenous extension tube when a force is applied to the intravenous extension tube and/or a source intravenous line connected to the intravenous extension tube.

FIG. 9C illustrates a releasable locking device 920 comprising a base 923, an aperture 926, and locks 929a-b. The releasable locking device 920 operates similar to the releasable locking devices 900 and 910 described in FIGS. 9A and 9B respectively.

FIG. 9D illustrates a releasable locking device 930 comprising locking mechanism 933 and an aperture 936. The releasable locking device 930 operates similar to the other releasable locking devices described in FIGS. 9A-C.

FIG. 9E illustrates a releasable locking device 940 comprising at least two apertures 943a-b. The intravenous extension tube passes through both apertures 943a and 943b. The releasable locking device 940 is configured to lock the intravenous extension tube by attaching the bottom portion 946 and the top portion 949 of the releasable locking device 940. The releasable locking device 940 operates similar to the other releasable locking devices described in FIGS. 9A-D.

FIG. 9F illustrates a releasable locking device 950 comprising a base 953, a holder 956, and an aperture 959. The base 953 comprises at least two locks configured to attach to an adhesive backing to be secured to a patient. The holder 956 is configured to receive an intravenous extension tube through the aperture 959. The holder 956 is also configured to detachably attach to the intravenous extension tube similar to the embodiments described in FIGS. 9A-9E.

FIG. 9G illustrates a releasable locking device 960 comprising an aperture 963, adjustable knob 965, arms 966a-b, and a locking mechanism 969. The locking mechanism 969 may be a spring lock mechanism, a snap lock mechanism, or any other mechanism that holds the arms 966a-b together and allows for the arms to detachably attach to an intravenous extension tube when a force is exerted on the intravenous extension tube. The arms 966a-b may be pushed together to activate the locking mechanism 969 to hold an intravenous extension tube using the aperture 963. In addition, a user of the releasable locking device 960 may manually detach the releasable locking device 960 from the intravenous extension tube by pushing together the arms 966a-b and releasing the locking mechanism 969. The adjustable knob 965 may be used to adjust the strength of the releasable locking device 960. For example, if a medical practitioner is preparing to insert a drain into a nephrostomy insertion point on a patient's back, the medical practitioner may adjust the level of force necessary to release the releasable locking device 960 from the intravenous extension tube using the adjustable knob 965. The medical practitioner may also make such an adjustment after considering numerous other factors, including the patient's age, the patient's sensitivity, the insertion point, the sophistication of drain equipment used, other stabilization devices used, and/or any other consideration that may assist in determining the force necessary to detach the releasable locking device 960 from the intravenous extension tube.

FIG. 9H illustrates a releasable locking device 970 comprising upper arms 973 and a base 976. Upper arms 973 may comprise locks 981a. The base 976 may comprise a holder 979 and locks 981b. An intravenous extension tube may be positioned in the holder 979 and secured by snap locking into the locks 981b. A user of the releasable locking device 970 may secure an intravenous extension tube by pushing down the upper arms 973 and snapping the locks 981a into the locks 981b. As illustrated in FIG. 9H, the releasable locking device 970 may comprise other snap lock portions that may operate to connect the upper arm 973 to the base 976 and the intravenous extension tube. As may be appreciated, an attachable portion of the intravenous extension tube may detachably attach to any portion of the base 976 and be secured by locking the upper arm 973 onto the base 976. The upper arms 973 may release the locks 981a-b and detach the intravenous extension tube when a force is applied to the intravenous extension tube.

As may be appreciated, the releasable locking device may be made of plastic, metal, or any material sufficient to detachably attach to an intravenous line or an electric cable. The intravenous extension tube may be made of plastic, metal, or any material sufficient to secure to an intravenous tube and/or an access device. The force exerted on the one or more lines of the substance delivery system may be an unexpected accidental force or may be a pre-defined configurable force that is scheduled to dislodge an infusion member of an access device from the patient. The releasable locking device may be automatically detached or manually detached from the intravenous extension tube.

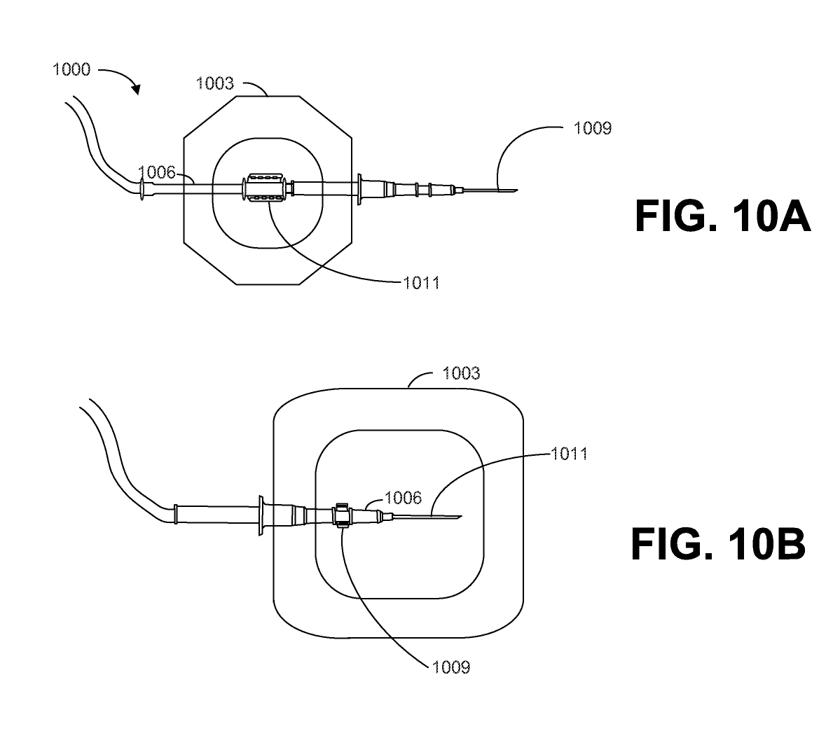

FIGS. 10A-B are drawings showing alternative embodiments of the intravenous line securing system depicted in FIGS. 6A-6B. FIG. 10A comprises the intravenous like securing system 1000 with at least one adhesive backing 1003, an intravenous extension tube 1006, and a needle 1011. The needle 1011 may alternatively be an insertion point of a drainage tube, electrical line, or any other insertion point of a line inserted into a patient's body. Adhesive backing 1003 comprises the releasable locking device 1009. As illustrated in FIG. 10A, the intravenous line securing system 1000 is secured onto the patient using the at least one adhesive backing 1003, a wrap, or other similar component. Adhesive backing 1003a secures the intravenous extension tube 1006 using the releasable locking device 1009. In addition, another adhesive backing 1003 may be used to secure the needle 1011 to the patient. If a pre-defined force is exerted on the intravenous line securing system 1000, the releasable locking device 1009 detaches from the intravenous extension tube 1006, but the needle 1011 and the adhesive backing 1003 may remain in place at the insertion site of the patient. The automatic detaching of the releasable locking device 1009 serves to prevent the needle 1011 from dislodging and injuring the patient.

FIG. 10B illustrates the embodiment of the intravenous line securing system 1000 using only one adhesive backing 1003 to securing the intravenous extension tube 1006 and the needle 1011. The adhesive backing 1003 comprises at least one releasable locking device 1009 configured to detachably attach to an attachable portion of the intravenous extension tube 1006. As illustrated in FIG. 10B, the intravenous extension tube 1006 is positioned closer to the needle 1011, allowing for the use of one adhesive backing 1003 to secure both the intravenous extension tube 1006 and the needle 1003. The releasable locking device 1009 operates to automatically detach from the intravenous extension tube 1006 when a pre-defined force is exerted upon the intravenous line securing system 1000.

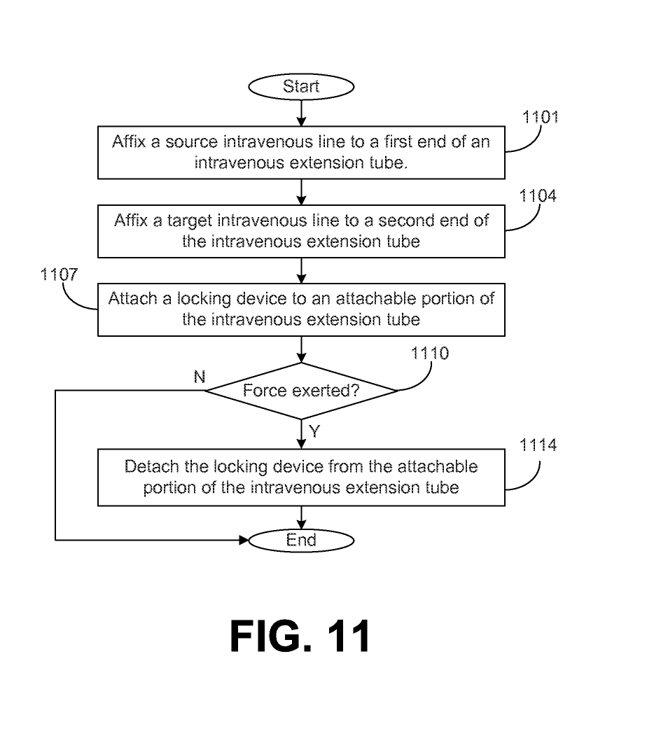

FIG. 11 is a flowchart illustrating one example of functionality implemented by a user of the substance delivery system comprising the line securing system according to various embodiments of the present disclosure. It is understood that the flowchart of FIG. 11 provides merely an example of many different types of functional arrangements that may be employed to implement the operation of the portion of the securing device as described herein. As an alternative, the flowchart of FIG. 11 may be viewed as depicting an example of steps and methods implanted by a patient or a doctor according to one or more embodiments.

Beginning with box 1101, the user of the line securing system, such as a medical practitioner, affixes a source intravenous line to a first end of an intravenous extension tube. The source intravenous line may channel solutions from a substance delivery bag to be directed toward the veins of a patient. Alternatively, the source intravenous line may be an electric cable used to monitor equipment or monitor vital patient information, including, but not limited to, cardiovascular readings. In box 1104, the user of the line securing system affixes a target intravenous line to a second end of the intravenous extension tube. The target intravenous line may be a portion of an intravenous tubing that channels solutions toward an injection site of the patient. Alternatively, the target intravenous line may be an access device, such as the ones described above in relation to FIGS. 2A and 2B. Alternatively the target intravenous line may also be an electric cable used to monitor equipment and/or patient information.

In box 1107, the user of the securing device system may attach the releasable locking device of an adhesive backing to an attachable portion of the intravenous extension tube. In box 1110, if a force is exerted on the intravenous extension tube, the source intravenous line, or the target intravenous line, the releasable locking device detaches the locking device from the attachable portion of the intravenous extension tube, as depicted in box 1114, after which the process ends. Alternatively, as shown in box 1110, if a force is not exerted on the intravenous extension tube, the source intravenous line, or the target intravenous line, then the releasable locking device remains attached to the attachable portion of the intravenous extension tube, after which the process ends.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

1. A system for stabilizing an intravenous line comprising:
an adhesive backing detachably attachable to a patient, the adhesive backing coupled to a releasable locking device, the releasable locking device comprising:
a lower semicircular arm,
an upper semicircular arm, the upper semicircular arm and the lower semicircular arm being hingedly interconnected at one end and having interlocking portions at another end, and
an aperture in between the lower semicircular arm and the upper semicircular arm, the lower semicircular arm and the upper semicircular arm configured to detachably attach to an intravenous extension tube, the intravenous extension tube disposed between a source intravenous line and a target intravenous line, the intravenous extension tube comprising:
a first portion of the intravenous extension tube coupled to the source intravenous line,
a second portion of the intravenous extension tube coupled to the target intravenous line, and
an attachable portion disposed in between the first portion and the second portion, the attachable portion being configured to couple to the lower semicircular arm and the upper semicircular arm of the releasable locking device
wherein the releasable locking device is configured to detach from the attachable portion in response to a pre-defined amount of force.

2. The system of claim 1, wherein the source intravenous line is configured to channel a liquid substance in an intravenous line bag.

3. The system of claim 1, wherein the target intravenous line is an access device.

4. The system of claim 3, wherein the access device is selected from a group consisting of a multi-port indwelling catheter, an arterial line, a central line, a peripherally inserted central catheter line, and a peripheral intravenous line.

5. A system for stabilizing an intravenous line comprising:
an adhesive backing detachably attachable to a patient, the adhesive backing comprising a releasable locking device configured to detachably attach to an intravenous extension tube,
wherein the releasable locking device is configured to detach the intravenous extension tube when a pre-defined force is exerted upon the intravenous extension tube.

6. The system of claim 5, wherein the releasable locking device comprises a lower semicircular arm and an upper semicircular arm, the upper semicircular arm and the lower semicircular arm being hingedly interconnected at one end and having interlocking portions at another end.

7. The system of claim 6, wherein the releasable locking device comprises an aperture in between the lower semicircular arm and the upper semicircular arm that holds the intravenous extension tube in the aperture.

8. The system of claim 5, wherein the releasable locking device comprises a lower semicircular arm wherein the intravenous extension tube detachably attaches to the lower semicircular arm of the clamp.

9. The system of claim 5, wherein the attachable portion is a plastic material and wherein a diameter of the attachable portion is larger than a diameter of the source intravenous line and larger than a diameter of the target intravenous line.

10. The system of claim 5, wherein intravenous extension tube comprises a first portion and a second portion, the second portion of the intravenous extension tube being more rigid than the first portion of the intravenous extension tube.

11. The system of claim 5, wherein source intravenous line is selected from a group consisting of a drainage tube, an electric line, and an intravenous tube.

12. The system of claim 5, wherein the target intravenous line is an access device.

13. The system of claim 12, wherein the access device is selected from a group consisting of a multi-port indwelling catheter, an arterial line, a central line, a peripherally inserted central catheter line, and a peripheral intravenous line.

14. The system of claim 5, wherein the bandage comprises three clamps.

15. A method for stabilizing an intravenous line comprising:
affixing a source intravenous line to a first end of an intravenous extension tube;
affixing a target intravenous line to a second end of the intravenous extension tube;
attaching at least one clamp of an adhesive backing to an attachable portion of the intravenous extension tube, the adhesive backing detachably attachable to a patient; and
releasing the at least one clamp from the attachable portion of the intravenous extension tube when a force has been exerted upon the intravenous extension tube.

16. The method of claim 15, wherein the intravenous extension tube further comprises a first portion and a second portion, wherein the attachable portion is affixed in between the first portion and the second portion.

17. The method of claim 16, wherein the first portion of the intravenous extension tube is coupled to the source intravenous line and the second portion of the intravenous extension tube is coupled to the target intravenous line.

18. The method of claim 17, wherein the second portion of the intravenous extension tube is more rigid than the first portion of the intravenous extension tube.

19. The method of claim 15, further comprising determining whether a force has been exerted on the source intravenous line

20. The method of claim 15, the at least one clamp comprises a base configured to hold and release the intravenous extension tube.
